(19) United States
(12) Patent Application Publication
(10) Pub. No.: US 2024/0307131 A1
(43) Pub. Date: Sep. 19, 2024

(54) Systems And Methods For An Image Guided Procedure

(71) Applicant: Mazor Robotics Ltd., Caesarea (IL)

(72) Inventors: Gad HARPAZ, Hod Hasharon (IL); Gillan M. Grimberg, Ramat Gan (IL); Leonid Kleyman, Atzmon-Segev (IL)

(73) Assignee: Mazor Robotics Ltd., Caesarea (IL)

(21) Appl. No.: 18/184,394

(22) Filed: Mar. 15, 2023

(51) Int. Cl. A61B 34/30; G06T 7/246; G06T 7/73

(52) U.S. Cl. CPC A61B 34/30; G06T 7/246; G06T 7/73; A61B 6/4458; A61B 2034/2065; A61B 2090/363; A61B 2090/376; G06T 2207/10116; G06T 2207/30004; G06T 2207/30204

(57) ABSTRACT

Registering a robotic coordinate system defined by a robotic system and a navigation coordinate system defined by a tracking system localizer. The registration includes: determining a robotic coordinate system between a first portion of the robotic system and a subject separate from the first portion of the robotic system; connecting a fiducial marker at a first known position relative to the robotic coordinate system and relative to the first portion of the robotic system; acquiring a fiducial image at an initial time with an imaging system of the fiducial marker; detecting the acquisition of the fiducial image with the imaging system at the initial time with a detector; and sending a command to determine or record an initial position of at least one of the first portion of the robotic system or the robotic coordinate system at the initial time.

### FIELD

The subject disclosure is directed to systems and methods for determining a timing of acquiring image data for correlating image-to-image data for surgical tracking and navigation system.

### BACKGROUND

This section provides background information related to the present disclosure, which is not necessarily prior art.

An instrument can be navigated relative to a subject for performing various procedures. For example, the subject can include a patient on which a surgical procedure is being performed. During a surgical procedure, an instrument can be tracked in an object or subject space. In various embodiments the subject space can be a patient space defined by a patient. The location of the instrument that is tracked can be displayed on a display device relative to an image of the patient.

The position of the patient can be determined with a tracking system. Generally, a patient is registered to the image, via tracking an instrument relative to the patient to generate a translation map between the subject or object space (e.g., patient space) and the image space. This often requires time during a surgical procedure for a user, such as a surgeon, to identify one or more points in the subject space and correlating, often identical points, in the image space.

After registration, the position of the instrument can be appropriately displayed on the display device while tracking the instrument. The position of the instrument relative to the subject can be displayed as a graphical representation, sometimes referred to as an icon on the display device.

### SUMMARY

This section provides a general summary of the disclosure, and is not a comprehensive disclosure of its full scope or all of its features.

Disclosed, according to various embodiments, is a system and method that is operable to register a robotic coordinate system and an image coordinate system. The system and method may include a robotic system having the robotic coordinate system defined relative to a first portion of the robotic system, wherein the robotic system is configured to be positioned relative to a subject. A detector on or proximate to the robotic system may be provided. A tracking system defining a navigation space having a navigation coordinate system may be included. A fiducial marker at a first known position in the robotic coordinate system relative to the first portion of the robotic system may also be included. Also, a processor system operable to execute instructions and/or perform various tasks may be included. The processor system may acquire a fiducial image at an initial time with an imaging system of the fiducial marker when the fiducial marker is at the first known position relative to the robotic coordinate system and relative to the first portion of the robotic system. The processor system may determine the initial time of the acquisition of the fiducial image with the imaging system with the detector. The processor system may also determine or record an initial position of at least one of the first portion of the robotic system or the robotic coordinate system at the initial time. Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is diagrammatic view illustrating an overview of a robotic system and a navigation system, according to various embodiments;

FIG. 2 is a detailed environmental view of a robotic system and a tracking system, according to various embodiments;

FIG. 3 is a detailed view of a robotic system with a snapshot tracking device, according to various embodiments;

FIG. 4 is a flow chart of a method of registering a robotic space to an image space;

FIG. 5 is a flow chart of a method in accordance with the preset disclosure for using a radiation detector for registering a first and second image; and

FIG. 6 is a flow chart further illustrating a method in accordance with the present disclosure for using the radiation detector for registering a first and second image.

Corresponding reference numerals indicate corresponding parts throughout the several views of the drawings.

### DETAILED DESCRIPTION

Example embodiments will now be described more fully with reference to the accompanying drawings.

The subject disclosure is directed to an exemplary embodiment of a surgical procedure on a subject, such as a human patient. It is understood, however, that the system and methods described herein are merely exemplary and not intended to limit the scope of the claims included herein. In various embodiments, it is understood, that the systems and methods may be incorporated into and/or used on non-animate objects. The systems may be used to, for example, to register coordinate systems between two systems for use on manufacturing systems, maintenance systems, and the like. For example, automotive assembly may use one or more robotic systems including individual coordinate systems that may be registered together for coordinated or consorted actions. Accordingly, the exemplary illustration of a surgical procedure herein is not intended to limit the scope of the appended claims.

Discussed herein, according various embodiments, are processes and systems for allowing registration between various coordinate systems. In various embodiments, a robotic or first coordinate system may be registered to an image coordinate system or space. A navigation space or coordinate system may then be registered to the robotic or first coordinate system and, therefore, be registered to the image coordinate system without being separately or independently registered to the image space. Similarly, the navigation space or coordinate system may be registered to the image coordinate system or space directly or independently. The robotic or first coordinate system may then be registered to the navigation space and, therefore, be registered to the image coordinate system or space without being separately or independently registered to the image space.

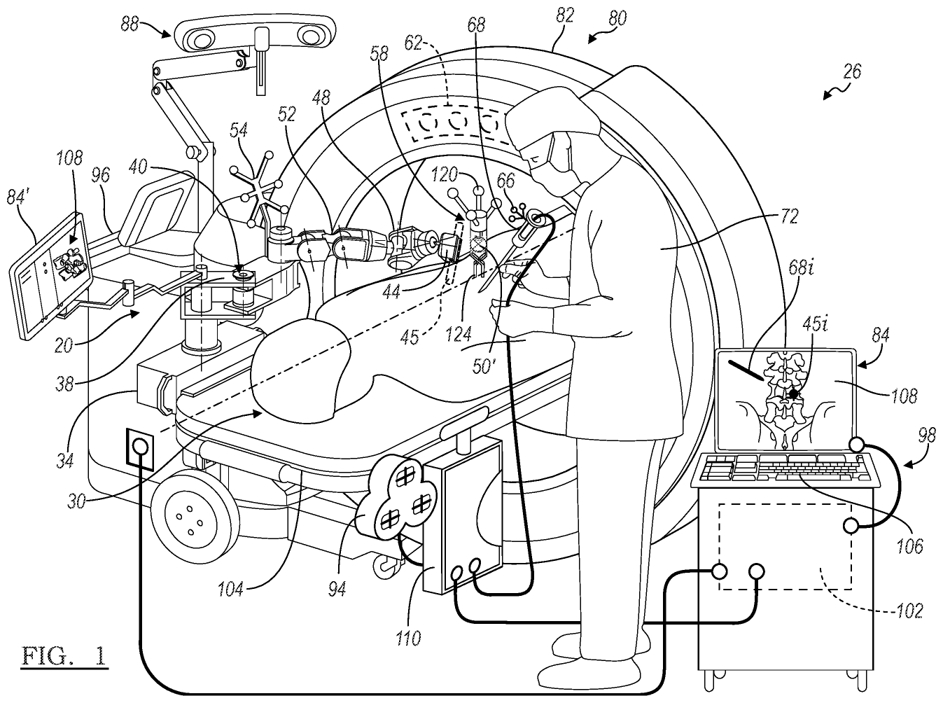

FIG. 1 is a diagrammatic view illustrating an overview of a procedure room or arena. In various embodiments, the procedure room may include a surgical suite in which may be placed a robotic system 20 and a navigation system 26 that can be used for various procedures. The robotic system 20 may include a Mazor X™ robotic guidance system, sold by Medtronic, Inc. The robotic system 20 may be used to assist in guiding selected instrument, such as drills, screws, etc. relative to a subject 30. As discussed herein, the robotic system 20 may be registered or correlated to a selected coordinate system such as via image-to image registration or correlation or similar methods, such as disclosed in U.S. Pat. No. 11,135,025, incorporated herein by reference. The robotic system 20 may include a mount 34 that fixes a portion, such as a robotic base 38, relative to the subject 30. The robotic system 20 may include one or more arms 40 that are moveable or pivotable relative to the subject 30, such as including an end effector 44 and mounted, such as moveably mounted to the base 38. The end effector may be any appropriate portion, such as a tube, guide, or passage member. The end effector 44 may be moved relative to the base 38 with one or more motors. The position of the end effector 44 may be known or determined relative to the base 38 with one or more encoders at one or more joints, such as a wrist joint 48 and/or an elbow joint 52 of the robotic system 20. At or near a selected portion of the robot 20, such as on the arm near one or more of the joints 48 and/or at or near the base 34 may be a sensor 50. The sensor 50 may be referred to as a detector, such as a x-ray detector. The sensors 50 may be used to sense when an image is acquired, such as with the imaging system 80.

The navigation system 26 can be used to track the location of one or more tracking devices, tracking devices may include a robot tracking device 54, a subject tracking device 58, an imaging system tracking device 62, and/or an tool tracking device 66. A tool 68 may be any appropriate tool such as a drill, forceps, or other tool operated by a user 72. The tool 68 may also include an implant, such as a spinal implant or orthopedic implant. It should further be noted that the navigation system 26 may be used to navigate any type of instrument, implant, or delivery system, including: guide wires, arthroscopic systems, orthopedic implants, spinal implants, deep brain stimulation (DBS) probes, etc. Moreover, the instruments may be used to navigate or map any region of the body. The navigation system 26 and the various instruments may be used in any appropriate procedure, such as one that is generally minimally invasive or an open procedure.

An imaging device 80 may be used to acquire pre-, intra-, or post-operative or real-time image data of a subject, such as the subject 30. It will be understood, however, that any appropriate subject can be imaged and any appropriate procedure may be performed relative to the subject. In the example shown, the imaging device 80 comprises an O-Arm® imaging device sold by Medtronic Navigation, Inc. having a place of business in Louisville, Colorado, USA. The imaging device 80 may have a generally annular gantry housing 82 in which an image capturing portion is moveably placed. The image capturing portion may include an x-ray source or emission portion and an x-ray receiving or image receiving portion located generally or as practically possible 180 degrees from each other and mounted on a rotor relative to a track or rail. The image capturing portion can be operable to rotate 360 degrees during image acquisition. The image capturing portion may rotate around a central point or axis, allowing image data of the subject 80 to be acquired from multiple directions or in multiple planes. The imaging device 80 can include those disclosed in U.S. Pat. Nos. 7,188,998; 7,108,421; 7,106,825; 7,001,045; and 6,940,941; all of which are incorporated herein by reference, or any appropriate portions thereof. In one example, the imaging device 80 can utilize flat plate technology having a 1,720 by 1,024 pixel viewing area. As is understood by on skilled in the art and mentioned herein, the imaging system 80 may be any appropriate imaging system such as the O-arm imaging system, a C-arm imaging system, etc.

The position of the imaging device 80, and/or portions therein such as the image capturing portion, can be precisely known relative to any other portion of the imaging device 80. The imaging device 80, according to various embodiments, can know and recall precise coordinates relative to a fixed or selected coordinate system. This can allow the imaging system 80 to know its position relative to the patient 30 or other references. In addition, as discussed herein, the precise knowledge of the position of the image capturing portion can be used in conjunction with a tracking system to determine the position of the image capturing portion and the image data relative to the tracked subject, such as the patient 30.

The imaging device 80 can also be tracked with a tracking device 62. The image data defining an image space acquired of the patient 30 can, according to various embodiments, be inherently or automatically registered relative to an object space. The object space can be the space defined by a patient 30 in the navigation system 26. The automatic registration can be achieved by including the tracking device 62 on the imaging device 80 and/or the determinable precise location of the image capturing portion. According to various embodiments, as discussed herein, imagable portions, virtual fiducial points and other features can also be used to allow for registration, automatic or otherwise. It will be understood, however, that image data can be acquired of any subject which will define subject space. Patient space is an exemplary subject space. Registration allows for a translation between patient space and image space.

The patient 80 can also be tracked as the patient moves with a patient tracking device, DRF, or tracker 58. Alternatively, or in addition thereto, the patient 30 may be fixed within navigation space defined by the navigation system 26 to allow for registration. As discussed further herein, registration of the image space to the patient space or subject space allows for navigation of the instrument 68 with the image data. When navigating the instrument 68, a position of the instrument 68 can be illustrated relative to image data acquired of the patient 30 on a display device 84. Various tracking systems, such as one including an optical localizer 88 or an electromagnetic (EM) localizer 92 can be used to track the instrument 68.

More than one tracking system can be used to track the instrument 68 in the navigation system 26. According to various embodiments, these can include an electromagnetic tracking (EM) system having the EM localizer 94 and/or an optical tracking system having the optical localizer 88. Either or both of the tracking systems can be used to tracked selected tracking devices, as discussed herein. It will be understood, unless discussed otherwise, that a tracking device can be a portion trackable with a selected tracking system. A tracking device need not refer to the entire member or structure to which the tracking device is affixed or associated.

It is further appreciated that the imaging device 80 may be an imaging device other than the O-Arm® imaging device and may include in addition or alternatively a fluoroscopic C-arm. Other exemplary imaging devices may include fluoroscopes such as bi-plane fluoroscopic systems, ceiling mounted fluoroscopic systems, cath-lab fluoroscopic systems, fixed C-arm fluoroscopic systems, isocentric C-arm fluoroscopic systems, 3D fluoroscopic systems, etc. Other appropriate imaging devices can also include MRI, CT, ultrasound, etc.

In various embodiments, an imaging device controller 96 may control the imaging device 80 and can receive the image data generated at the image capturing portion and store the images for later use. The controller 96 can also control the rotation of the image capturing portion of the imaging device 80. It will be understood that the controller 96 need not be integral with the gantry housing 82, but may be separate therefrom. For example, the controller may be a portions of the navigation system 26 that may include a processing and/or control system 98 including a processing unit or processing portion 102. The controller 96, however, may be integral with the gantry 82 and may include a second and separate processor, such as that in a portable computer.

The patient 30 can be fixed onto an operating table 104. According to one example, the table 104 can be an Axis Jackson® operating table sold by OSI, a subsidiary of Mizuho Ikakogyo Co., Ltd., having a place of business in Tokyo, Japan or Mizuho Orthopedic Systems, Inc. having a place of business in California, USA. Patient positioning devices can be used with the table, and include a Mayfield® clamp or those set forth in commonly assigned U.S. patent application Ser. No. 10/405,068 entitled “An Integrated Electromagnetic Navigation And Patient Positioning Device”, filed Apr. 1, 2003 which is hereby incorporated by reference.

The position of the patient 30 relative to the imaging device 80 can be determined by the navigation system 26. The tracking device 62 can be used to track and locate at least a portion of the imaging device 80, for example the gantry or housing 82. The patient 30 can be tracked with the dynamic reference frame 58, as discussed further herein. Accordingly, the position of the patient 30 relative to the imaging device 80 can be determined. Further, the location of the imaging portion can be determined relative to the housing 82 due to its precise position on the rail within the housing 82, substantially inflexible rotor, etc. The imaging device 80 can include an accuracy of within 10 microns, for example, if the imaging device 80 is an O-Arm® imaging device sold by Medtronic Navigation, Inc. having a place of business in Louisville, Colorado. Precise positioning of the imaging portion is further described in U.S. Pat. Nos. 7,188,998; 7,108,421; 7,106,825; 7,001,045; and 6,940,941; all of which are incorporated herein by reference,

According to various embodiments, the imaging device 80 can generate and/or emit x-rays from the x-ray source that propagate through the patient 30 and are received by the x-ray imaging receiving portion. The image capturing portion generates image data representing the intensities of the received x-rays. Typically, the image capturing portion can include an image intensifier that first converts the x-rays to visible light and a camera (e.g. a charge couple device) that converts the visible light into digital image data. The image capturing portion may also be a digital device that converts x-rays directly to digital image data for forming images, thus potentially avoiding distortion introduced by first converting to visible light.

Two dimensional and/or three dimensional fluoroscopic image data that may be taken by the imaging device 80 can be captured and stored in the imaging device controller 96. Multiple image data taken by the imaging device 80 may also be captured and assembled to provide a larger view or image of a whole region of a patient 30, as opposed to being directed to only a portion of a region of the patient 30. For example, multiple image data of the patient's 30 spine may be appended together to provide a full view or complete set of image data of the spine.

The image data can then be forwarded from the image device controller 96 to the navigation computer and/or processor system 102 that can be a part of a controller or work station 98 having the display 84 and a user interface 106. It will also be understood that the image data is not necessarily first retained in the controller 96, but may also be directly transmitted to the work station 98. The work station 98 can provide facilities for displaying the image data as an image 108 on the display 84, saving, digitally manipulating, or printing a hard copy image of the received image data. The user interface 106, which may be a keyboard, mouse, touch pen, touch screen or other suitable device, allows the user 72 to provide inputs to control the imaging device 80, via the image device controller 96, or adjust the display settings of the display 84. The work station 98 may also direct the image device controller 96 to adjust the image capturing portion of the imaging device 80 to obtain various two-dimensional images along different planes in order to generate representative two-dimensional and three-dimensional image data.

With continuing reference to FIG. 1, the navigation system 26 can further include the tracking system including either or both of the electromagnetic (EM) localizer 94 and/or the optical localizer 88. The tracking systems may include a controller and interface portion 110. The controller 110 can be connected to the processor portion 102, which can include a processor included within a computer. The EM tracking system may include the STEALTHSTATION® AXIEM™ Navigation System, sold by Medtronic Navigation, Inc. having a place of business in Louisville, Colorado; or can be the EM tracking system described in U.S. patent application Ser. No. 10/941,782, filed Sep. 15, 2004, and entitled “METHOD AND APPARATUS FOR SURGICAL NAVIGATION”; U.S. Pat. No. 5,913,820, entitled “Position Location System,” issued Jun. 22, 1999; and U.S. Pat. No. 5,592,939, entitled “Method and System for Navigating a Catheter Probe,” issued Jan. 14, 1997; all of which are herein incorporated by reference. It will be understood that the navigation system 26 may also be or include any appropriate tracking system, including a STEALTHSTATION® TREON® or S7™ tracking systems having an optical localizer, that may be used as the optical localizer 88, and sold by Medtronic Navigation, Inc. of Louisville, Colorado. Other tracking systems include an acoustic, radiation, radar, etc. The tracking systems can be used according to generally known or described techniques in the above incorporated references. Details will not be included herein except when to clarify selected operation of the subject disclosure.

Wired or physical connections can interconnect the tracking systems, imaging device 80, etc. Alternatively, various portions, such as the instrument 68 may employ a wireless communications channel, such as that disclosed in U.S. Pat. No. 6,474,341, entitled “Surgical Communication Power System,” issued Nov. 5, 2002, herein incorporated by reference, as opposed to being coupled directly to the controller 110. Also, the tracking devices 62, 66, 54 can generate a field and/or signal that is sensed by the localizer(s) 88, 94.

Various portions of the navigation system 26, such as the instrument 68, and others as will be described in detail below, can be equipped with at least one, and generally multiple, of the tracking devices 66. The instrument can also include more than one type or modality of tracking device 66, such as an EM tracking device and/or an optical tracking device. The instrument 68 can include a graspable or manipulable portion at a proximal end and the tracking devices may be fixed near the manipulable portion of the instrument 68.

Additional representative or alternative localization and tracking system is set forth in U.S. Pat. No. 5,983,126, entitled “Catheter Location System and Method,” issued Nov. 9, 1999, which is hereby incorporated by reference. The navigation system 26 may be a hybrid system that includes components from various tracking systems.

According to various embodiments, the navigation system 26 can be used to track the instrument 68 relative to the patient 30. The instrument 68 can be tracked with the tracking system, as discussed above. Image data of the patient 30, or an appropriate subject, can be used to assist the user 72 in guiding the instrument 68. The image data, however, is registered to the patient 30. The image data defines an image space that is registered to the patient space defined by the patient 30. The registration can be performed as discussed herein, automatically, manually, or combinations thereof.

Generally, registration allows a translation map to be generated of the physical location of the instrument 68 relative to the image space of the image data. The translation map allows the tracked position of the instrument 68 to be displayed on the display device 84 relative to the image data 108. A graphical representation 68i, also referred to as an icon, can be used to illustrate the location of the instrument 68 relative to the image data 108.

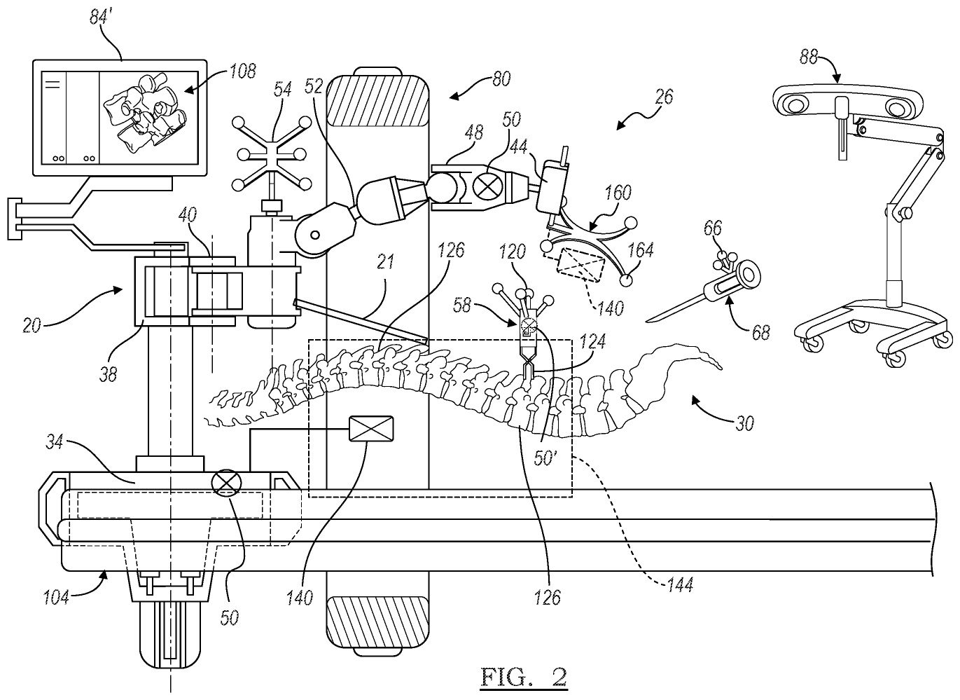

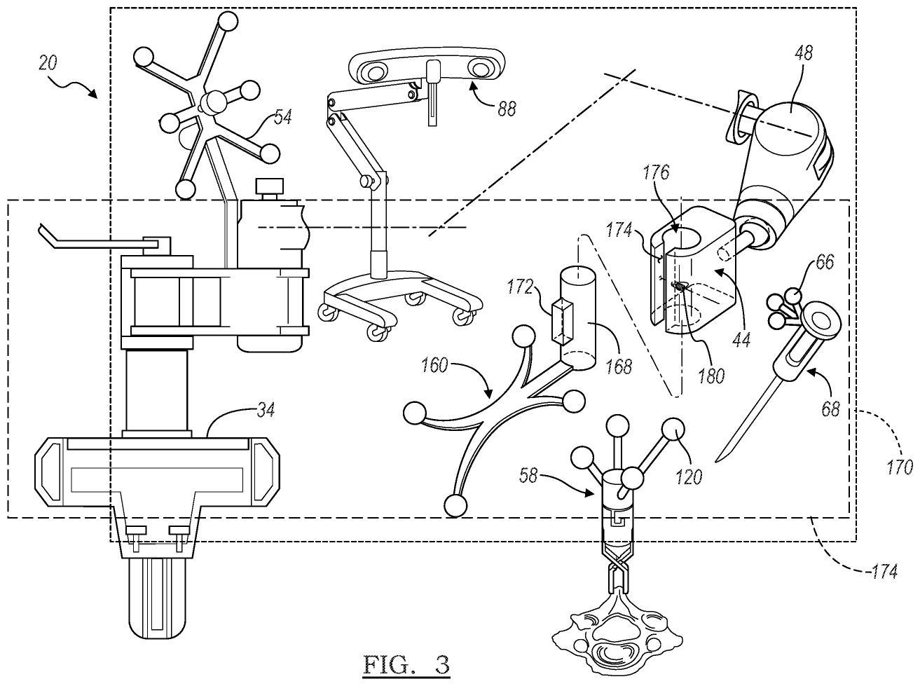

With continuing reference to FIG. 1 and additional reference to FIG. 2 and FIG. 3, a subject registration system or method can use the tracking device 58. The tracking device 58 may include portions or members 120 that may be trackable, but may also act as or be operable as a fiducial assembly. The fiducial assembly 120 can include a clamp or other fixation portion 124 and the imagable fiducial body 120. It is understood, however, that the members 120 may be separate from the tracking device 58. The fixation portion 124 can be provided to fix any appropriate portion, such as a portion of the anatomy. As illustrated in FIGS. 1 and 2, the fiducial assembly 120 can be interconnected with a portion of a spine 126 such as a spinous process 130. Associated with the tracking device 58, including with and/or in addition to the members 120 and/or the fixation portion 124, may be the sensor and/or an alternative sensor 50′. The sensor 50′ may operate in a manner similar or identical to the sensor 50 as discussed above and herein. It is understood, however, that the sensor 50 may be located at any appropriate position to sense when an image is acquired, such as on the table 104, at or on the imaging system 80, etc. It is understood that one and/or only one sensor may be used and/or more than one sensor may be used at any appropriate position.

The fixation portion 124 can be interconnected with a spinous process 130 in any appropriate manner. For example, a pin or a screw can be driven into the spinous process 130. Alternatively, or in addition thereto, a clamp portion 124 can be provided to interconnect the spinous process 130. The fiducial portions 120 may be imaged with the imaging device 80. It is understood, however, that various portions of the subject (such as a spinous process) may also be used as a fiducial portion.

In various embodiments, when the fiducial portions 120 are imaged with the imaging device 80, image data is generated that includes or identifies the fiducial portions 120. The fiducial portions 120 can be identified in image data automatically (e.g. with a processor executing a program), manually (e.g. by selection an identification by the user 72), or combinations thereof (e.g. by selection an identification by the user 72 of a seed point and segmentation by a processor executing a program). Methods of automatic imagable portion identification include those disclosed in U.S. Pat. No. 8,150,494 issued on Apr. 3, 2012, incorporated herein by reference. Manual identification can include selecting an element (e.g. pixel) or region in the image data wherein the imagable portion has been imaged. Regardless, the fiducial portions 120 identified in the image data can be used as fiducial points or positions that can be used to register the image data or the image space of the image data with patient space.

In various embodiments, to register an image space or coordinate system to another space or coordinate system, such as a navigation space, the fiducial portions 120 that are identified in the image 108 may then be identified in the subject space defined by the subject 30, in an appropriate manner. For example, the user 72 may move the instrument 68 relative to the subject 30 to touch the fiducial portions 120, if the fiducial portions are attached to the subject 30 in the same position during the acquisition of the image data to generate the image 108. It is understood that the fiducial portions 120, as discussed above in various embodiments, may be attached to the subject 30 and/or may include anatomical portions of the subject 30. Additionally, a tracking device may be incorporated into the fiducial portions 120 and they may be maintained with the subject 30 after the image is acquired. In this case, the registration or the identification of the fiducial portions 120 in a subject space may be made. Nevertheless, according to various embodiments, the user 72 may move the instrument 68 to touch the fiducial portions 120. The tracking system, such as with the optical localizer 88, may track the position of the instrument 68 due to the tracking device 66 attached thereto. This allows the user 72 to identify in the navigation space the locations of the fiducial portions 120 that are identified in the image 108. After identifying the positions of the fiducial portions 120 in the navigation space, which may include a subject space, the translation map may be made between the subject space defined by the subject 30 in a navigation space and the image space defined by the image 108. Accordingly, identical or known locations allow for registration as discussed further herein.

In various embodiments, the robotic system 20 may be interconnected with it the subject 30, such as with a fixation or holding member 21. The holding number 21 may extend from any appropriate portion of the robotic system 20, such as from or near the base 34, the fixed portion 38, and/or the robotic arm 40. The holding member 21 may connect at least a portion of the robotic system 20 relative to the subject 30 (e.g., with a fixation member such as a screw or clamp). As discussed above the tracking device 54 may be associated with the robotic system 20. Therefore, the robotic system 20 may also operate as a patient tracker, similar to the fiducial or tracker 58, relative to the subject 30. As the robotic system 20 may be connected (which may include a selected fixation), at least in part, relative to the subject 30 the tacking portion 54 may be used to track a portion of the subject 30. Various embodiments the holding member 21 may be connected directly to the portion of the subject 30 in which a procedure is occurring and/or relative to the portion on which procedures occurring and the portion may generally be immovable relative to the procedure portion of the subject 30. Thus, the patient tracker 58 need not be connected to the subject 30, particularly when the holding number 21 is connected to the subject 30 and to the robotic system 20. It is understood, however, that to the patient tracker 58 may be the only tracker connected the patient 30 to be tracked with the navigation system. The robotic system 20 may be separately tracked with the navigation system relative to the subject 30. Further the holding number 21 may be the only portion fixed to the patient 30 and the tracking device 54 may be tracked. It is understood, however, that while only one or the other may be connected, both may also be connected to the subject 30, according to various embodiments.

During registration, a translation map is determined between the image data coordinate system of the image data such as the image 108 and the patient space defined by the patient 30. Once the registration occurs, the instrument 68 can be tracked with the tracking system that is registered to the image data to allow an identification and illustration of a position of the tracked instrument 68 as an icon superimposed on the image data. Registration of the image 108 (or any selected image data) to the subject 30 may occur at any appropriate time.

After the registration of the image space to the patient space, the instrument 68 can be tracked relative to the image 108. As illustrated in FIG. 1, the icon 68i representing a position (which may include a 6 degree of freedom position (including 3D location and orientation)) of the instrument 68 can be displayed relative to the image 108 on the display 84. Due to the registration of the image space to the patient space, the position of the icon 68i relative to the image 108 can substantially identify or mimic the location of the instrument 68 relative to the patient 30 in the patient space. As discussed above, this can allow a navigated procedure to occur.

The robotic system 20 having the robotic system coordinate system may be registered to the navigation space coordinate system, as discussed herein, due to the reference tracking device 54 (e.g. if fixed to a known position on or relative to the robotic system 20) and/or due to the tracking of the snapshot tracking device 160. The snapshot tracking device 160 may include one or more trackable portions 164 that may be tracked with the localizer 88 or any appropriate localizer (e.g. optical, EM, radar). It is understood, however, that any appropriate tracking system may be used to track the snapshot tracking device 160. A fixed reference tracking device may also be positioned within the navigation space. The fixed navigation tracker may include the patient tracker 58 which may be connected to the patient 30 and/or the robot tracker 54 that may be fixed to the base 34 of the robotic system 20. The reference tracker, therefore, may be any appropriate tracker that is positioned relative to the snapshot tracker 160 that is within the navigation coordinate space during the registration period. For the discussion herein the robot tracker 54 will be referred to however, the patient tracker 58 may also be used as the reference tracker. Further, reference tracker may be positioned within the coordinate system at any position relative to the snapshot tracker 160 as long as the snapshot tracker 160 may be tracked relative to the reference tracker.

In various embodiments, the snapshot tracker 160 may be positioned at a known position relative to the end effector 44. For example, the snapshot tracker 160, as illustrated in FIG. 3, which includes the trackable portions 164, extends from a rod or connection member 168. The connection member 168 may include a keyed portion, such as a projection 172 that may engage a slot 174 of the end effector 44. The end effector 44 may form or define a cannula or passage 176 that may engage the connector 168. The connector 168 may be positioned within the passage 176 of the end effector 44. The connector 168 may then be fixed to the end effector 44, such as with a fixation member including a set screw or clamping of the end effector 44, such as with a set screw or clamping member 180. The projection 172 may engage within the slot 174 to fix the snapshot tracker 160 rotationally relative to the end effector 44. The connector 168 positioned within the passage 176 and locked in place with the set screw 180 may then rigidly fix the snapshot tracking device 160 relative to the end effector 44. Thus, the position of the snapshot tracker 160 relative to the end effector 44 may be fixed.

The localizer 88 may then view or determine a position of the snapshot tracking device 160 relative to the reference tracking device 54 and or the reference tracking device 58. As the localizer 88 defines or may be used to define the navigation space, determining or tracking a position of the snapshot localizer 160 relative to the reference frame 54 may be used to determine a relationship between a position within the navigation space and the robotic space of the end effector 44.

With continuing reference to FIG. 3, therefore, the navigation space defined by the localizer 88 may include the full navigation space 170 which may include portions relative to the subject, such as the subject tracker 58 and other portions that may be moved therein, such as the instrument 68. The robotic registration space may be smaller and may include a robotic registration space 174 that may include the reference frame 54 and the snapshot tracker 160. As discussed above, however, the robot registration navigation space may include the snapshot tracker 160 and the patient tracker 58 for registration. Accordingly, the exemplary registration navigation space 174 is merely for the current discussion. As discussed herein, both the robotic reference tracker 54 and the patient tracker 58 need not be used simultaneously. This is particularly true when the patient 30 is fixed in space, such as fixed relative to the robotic system 20.

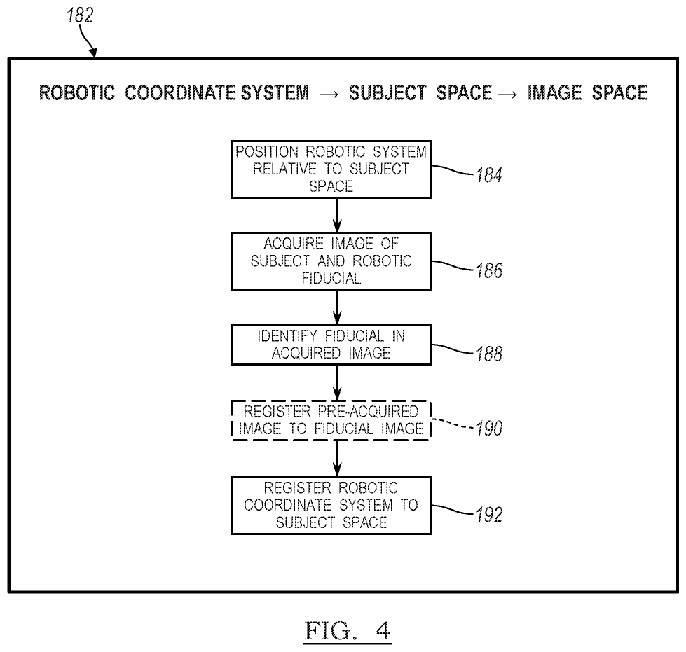

With continuing reference to FIG. 2 and FIG. 3, and additional reference to FIG. 4, a process or method of coordinating or registering a robotic coordinate system of the robotic system 20 to a navigation space or navigation coordinate system of the navigation system 26 is described. The registration may include various portions or sub-parts, as discussed herein. The various parts may occur in any appropriate order, and the order discussed herein is merely exemplary. The co-registration may further allow only one coordinate system of the robotic or navigation to be registered to a third coordinate system, such as an image coordinate system, but allow the registration of the other to the third coordinate system.

The robotic system 20, as discussed above, is positioned relative to the subject 30 for various portions of a procedure. In various embodiments, the robotic system 20 may be registered to the subject 30 and to the image 108 of the subject 30, that may be displayed on the display device 84 and/or a second or auxiliary display device 84′ that may be movable relative to the robotic system 20. The imaging system 80, or any appropriate imaging system, may be used to image the subject 30. The image may include a portion of the subject, such as one or more of the vertebrae 126 and a fiducial or robotic fiducial array 140 that may be fixed to the robotic system 20. The robotic fiducial 140 may be fixed to a selected portion of the robotic system 20, such as to the base 34 and/or the fixed portion 38. The robotic fiducial 140 may also and/or alternatively be connected to the end effector 44 (illustrated in phantom in FIG. 2). The robotic fiducial 140 may be positioned relative to the subject 30 for acquisition of images such that the fiducial 140 is apparent in the images. Upon acquisition of the image of the robotic fiducial 140 and portions of the subject 30, such as the vertebrae 126, the position of the robotic fiducial 140 relative to the vertebrae 126 may be determined. If the robotic fiducial 140 is fixed to the robotic system 20, the robotic coordinate system may be determined relative to the subject space coordinate system. In various embodiments, if the fiducial 140 is connected to the end effector 44, the known position of the end effector in the robotic coordinate system allows for image registration to the robotic coordinate system of the robotic system 20. With continuing reference to FIG. 2 and additional reference to FIG. 4, the robotic coordinate system may be registered to a subject space or coordinate system in the method 182 as described.

Generally, the registration may include positioning the robotic system 20 relative to a subject space in block 184. Positioning of the robotic system 20 relative to the subject space may include positioning the robotic system 30 relative to the subject, as illustrated in FIG. 2. Further, positioning of the robotic system 20 may include positioning or removably positioning the robotic fiducial 140 relative to the subject 30. The robotic fiducial 140 may be removably placed in a position relative to the robotic system 20 for various procedures and may be substantially positioned in the same position for different or subsequent procedures. With the subject 30 positioned relative to the robotic system 20, fiducial images may be acquired of the subject 30 and the robotic fiducial 140 with the imaging system 80 in block 186. The acquisition of the fiducial images in block 186 allows for image data to be acquired of the subject 30, such as with the vertebrae 126, and the fiducial 140.

As illustrated in FIG. 2, the robotic system 20 may include the sensor 50 that may sense radiation (e.g., x-ray radiation), or any other suitable sensor configured to detect when the image of the subject 30 and robotic fiducial 140 is taken at block 186. The radiation sensor 50 may be located at any suitable position on or near the robotic system 20, such as proximate to the end effector 44 and/or near the var 34 as illustrated in FIG. 2. The sensor 50 may be positioned at any appropriate positions on or relative to the robot 20 to sense that an image is acquired. It is understood, one or more of the sensors 50 may be provided with the robotic system 20. As noted above, the radiation sensor 50. 50′ may be positioned at any appropriate position relative the imaging system 26.

The radiation sensor 50 is in communication with the processor 102 of the workstation 98 and/or the controller 96 of the robotic system 20. Upon detection of radiation, such as with the sensor 50, 50′, the sensor 50 is configured to transmit a notification to the processor 102 and/or the controller 96. The notification may be any appropriate signal and may be transmitted with any appropriate connection, e.g., wireless or wired. The discussion herein of the sensor 50 is understood to generally refer to any appropriate radiation sensor such as the sensor 50′ and/or a sensor placed at any other position configured to sense radiation from the imaging system 26.

Upon receipt of the notification, the processor 102 and/or the controller 96 is configured to save the position of the robotic arm 40 (including the Euclidian positions and orientations of the fiducial 140 and the robotic arm 40, including the end effector 44) at the same time that the image was acquired in block 186 so that the processor 102 and/or the controller 96 know the precise location of the robotic system 20 during registration of the robotic coordinate system to the subject space described at block 192.

That is the sensor 50 may sense the moment, such as a precise time, when the image is acquired of the fiducial 140. The precise time of the acquisition of the image of the fiducial 140 may be used to assist in making a registration of the robotic system 20 reference frame to the image reference frame, such as of the image 108. As discussed above, the image required of the fiducial 140 may be used for registration of a coordinate reference frame of the robotic arm 20 and the pre-acquired image, which may be any appropriate type of image. They pre-acquired image may be a two- and/or three-dimensional image (such as a CT image, MRI image, or the like). The image acquired of the fiducial 140 may also be any appropriate type of image, such as a two-dimensional fluoroscopic image, a two-dimensional image acquired with the O-Arm® imaging system, a cone beam image required with the O-Arm® imaging system, or other appropriate image data. Regardless, the sensor 50 allows for an immediate notification that the image of the fiducial 140 has been acquired and/or a precise time as to the acquisition of the image of the fiducial 140.

As a result, any movement of the robotic arm 40 following the acquisition of the image of the fiducial for registration but before final registration and/or during the registration process will not negatively impact the registration at block 192. In other words, as the pose of the robotic system 20 is known as it can be recalled and/or determined at the time at the sensor 50 senses the image acquisition (e.g., via radiation detection), movement following the image acquisition will not be used to perform the registration.

In addition, sensing radiation with the sensor 50. 50′ may be used to generate the signal for any appropriate purpose. The signal may be used to determine or save a pose of the imaging system, the tracked pose of the patient tracker 58, the robotic arm 40, end effector 44, or any other appropriate portion. Thus, determining or saving the pose of the end effector 44 and/or the robotic arm is merely exemplary.

Also, the sensor 50 will eliminate any need to manually notify the processor 102 and/or the controller 96, such as by way of a manual mouse click, touch, keystroke, etc., that the image of the subject 30 and the robotic fiducial 140 is being taken at block 186. This advantageously frees resources in the procedure room because procedure room personnel do not have to devote attention to manually notify the processor 102 and/or the controller 96 that the image of the subject 30 and the fiducial 140 is being taken. This may reduce the need for confirmation of determining a pose of the robotic system during the image acquisition. Further, communication amongst personnel in the procedure room need not occur during the registration image acquisition. All may make the registration more automatic and efficient.

After acquisition of the robotic fiducial image in block 186, identifying of the robotic fiducial 140 in the acquired fiducial images occurs in block 188. Identification of the robotic fiducial in the robotic fiducial images may be manual, automatic, or a combination of automatic and manual. For example, the user may identify the robotic fiducial in the image a selected automatic system may segment the fiducials from the fiducial images, or the user may identify a seed pixel or voxel or multiple seed pixels or voxels and the processor system may further segment the fiducial system.

In various embodiments, the acquired images in block 186 may be used for planning and/or performing a procedure. For example, the imaging system 80 may acquire image data sufficient for a selected procedure. Thus, the images acquired in block 186 may be used for planning and navigating a selected procedure relative to the subject 30. The image data may include two-dimensional image data, reconstructed three-dimensional image data, and/or image data acquired over time to illustrate movement of motion of the subject (which may be acquired in 2D or 3D).

In various embodiments, however, the fiducial image acquired in block 186 may be optionally registered to other-time or pre-acquired images in block 190, such as an MRI or a computed tomography scan of the subject 30 prior to the acquisition of the fiducial images in block 186. The pre-acquired images may be acquired at any appropriate time prior to the acquisition of the fiducial images in block 186. It is understood, however, that the images may be acquired after the fiducial images and may be registered to the fiducial images in a similar manner as discussed herein. The registration of the fiducial images to the pre-acquired images may occur in any appropriate manner such as segmentation of selected vertebrae, identification in registration of selected fiducial elements in the images (e.g. anatomical fiducial portions and/or positioned or implanted fiducial members) or other appropriate procedures. Generally, the Mazor X® Robotic System may generally allow for registration of a pre-acquired image to the fiducial images and may be appropriate for registering the fiducial images in block 186 to the pre-acquired images in the registration of the pre-acquired image to the fiducial image in block 190.

The robotic coordinate system may also be registered to the subject space in block 192 with the identification of fiducials in the image in block 188 and the registration. The robotic fiducial 140, imaged with the fiducial images in block 186, is positioned in a known position relative to the robotic system 20, such as the base 34 and/or with the known position of the end effector 44 in the robotic coordinate system. The robotic coordinate system that is defined by the robotic system 20 relative to the base 34 and/or the fixed portion 38 may, therefore also, be pre-determined or known relative to the robotic fiducial 140 as the robotic fiducial 140 is fixed relative to the robotic system 20. When position with the end effector 44, the position of the robotic fiducial 140 is known in the robotic coordinate system by tracked (e.g. robotic system tracking) movement of the end effector 44. The fiducial image acquired in block 186 may also assist in defining the patient space relative to which the robotic system 20, particularly the end effector movable portion 44, may move is also then known. As discussed above, the end effector 44 moves in the robotic coordinate system due to the robotic tracking system that may include various mechanisms, such as encoders at the various movable portions, such as the wrist 48 or elbow 52, of the robotic system 20. If the fiducial images in block 186 are the images for performing the procedure, such as for navigation and may the displayed image 108, the registration may be substantially automatic as the subject 30 may be substantially fixed relative to the robotic system 20 (e.g. with a fixation ember extending from the base 38) and connected to the subject 30, such as the vertebrae 126.

Accordingly the robotic coordinate system can be registered to the subject space and/or image space according to the method 182. Given the registration of the robotic coordinate system to the image space the robotic, coordinate system registration may be used to determine a position of the end effector 44 and/or a member positioned through or with the end effector 44, relative to the image 108. Accordingly, the image 108 may be used to display a graphical representation, such as a graphical representation of the member or instrument 45 as an icon 45i superimposed or superimposed relative to the image 108.

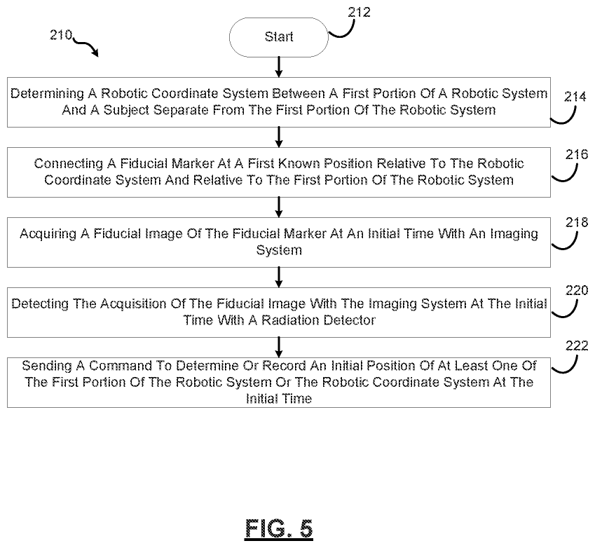

FIG. 5 illustrates an exemplary method 210, according to various embodiments of the present disclosure, for registering two images, such as CT to fluoroscopic imaging registration. The method 210 may be performed by the processor 102, or any other suitable processor. The method 210 is described herein as being performed by the processor 102 for exemplary purposes only. The method 210 may be used to assist in a registration, ensure accuracy of a registration, and/or increase efficiency of a registration by limiting or eliminating manual involvement.

The method 210 starts at block 212, and at block 214 the method 210 determines a robotic coordinate system between a first portion of the robotic system 20 and the subject 30. The robotic system 20 includes portions, such as an arm or end effector 44 that may be the first portion, which is separate from the subject 30 and generally moveable relative to the subject 30. The robotic coordinate system may be determined, as noted above, and may be defined by the robotic system 20 and/or relative to a portion thereof such as the base 34 to which the fiducial 140 may be fixed or positioned.

At block 216, the method 210 includes connecting the robotic fiducial 140 at a first known position relative to the robotic coordinate system and relative to the first portion of the robotic system 20. The robotic fiducial 140 may be connected (e.g., permanently or temporarily fixed) to a selected portion of the robotic system 20, such as to the base 34 and/or the fixed portion 38. The robotic fiducial 140 may also and/or alternatively be connected to the end effector 44 (illustrated in phantom in FIG. 2). The fiducial 140 may be imaged in a selected imaging modality, such as with the imaging system 80. The fiducial 104 may be imaged simultaneously with one or more portions of the subject 30. Thus, an image data may include both at least a portion of the fiducial 140 and the subject 30.

At block 218, the method 210 includes acquiring a fiducial image of the robotic fiducial 140 at an initial time (which may also be referred to as fiducial image time) with the imaging system 80. The image may be acquired automatically, such as when the robotic system 20 is at a selected pose and/or based on manual input. For example, the user may include that the fiducial image is to be acquired.

Following the fiducial image acquisition, at block 220, the processor 102 detects the acquisition of the fiducial image with the imaging system 80 at the initial time via the sensor 50. As noted above, the sensor 50, according to various embodiments, may transmit a signal. The signal from the sensor 50 may be based on a detection of radiation. The detection of radiation may be due to the acquisition of the fiducial image. In various embodiments, the sensor 50 will only generate a signal when the fiducial image is acquired (e.g., when radiation is detected at the sensor due to the imaging system 80). In various embodiments the sensor 50 may generate or send the signal any time an image is acquired, and the processor 102 may determine the time stamp for an image that is to be used as the registration image and detemir or recall a pose of the robotic system 20 at the same time.

At block 222, the processor 102 determines or records an initial position (also referred to as the fiducial position) of at least one of the first portion of the robotic system or the robotic coordinate system at the initial time. When determining the initial position (i.e., fiducial position), the robotic system 20 may include a various system to determine a pose of one or more portions of the robotic system 20. As noted above, various joints may have sensors relative thereto determine a pose of the various joints. Also, sensors may be used to determine a pose of the end effector 44 relative to the other portions of the robotic system 20, such as the base 34. The initial position may be the pose or position of any portion of the robotic system 20 that may be used by the navigation system to determine a registration of the robotic coordinated reference frame relative to the coordinate frame of the pre-acquired image data that may be based upon the fiducial image. Thus, the processor 102 may determine or recall the initial pose of the robotic system at the time of the acquisition of the fiducial image. The pose of the robotic system may be acquired immediately at the time of the acquisition of the signal from the sensor 50, such as in real time (e.g., instantaneously).

As discussed above, the robotic system 20 may be held relative to the subject 30 with the holding member 21. As the holding member may fix a portion of the robotic system 20 relative to the subject 30, a pose of the robotic system 20 relative to the subject 30 may be determined at any appropriate time. Thus, the sensor 50 may sense of the radiation and be used to identify or collect information regarding a pose of the robotic system 20, including the robotic arm 40, relative to the subject 30. As the robotic system 20 may also be held relative to the subject 30 and the fiducial her patient tracker 58 may not be used, according to various embodiments.

According to various embodiments, such as the method 210 and the system discloses herein, the pose of the robotic system 20 may be determined substantially instantaneously with the acquisition of the fiducial image based upon the sensed acquisition of the image with the sensor 50. As discussed above this may be based upon sensing radiation with of the sensor 50. In various embodiments, the pose of the robotic system 20 may be determined based upon the signal from the sensor 50. For example, the pose of the robotic system 20 may be determined based upon a time that relates to the acquisition of the fiducial image. In other words, the pose of the robotic system 20 over time may be determined or recorded and timestamped. The time stamp that relates to the acquired of the fiducial image may be matched based on the signal form the sorn 50. Also, the signal from the sensor 50 may be used to record a pose of the robotic system 50. Regardless of the sequence, however, the pose of the robotic system 20 at the time of the acquisition of the fiducial image may be based upon the signal sent from the sensor 50. Substantially the position of the robotic system 20 may be known precisely at the instant of the acquisition of the fiducial image. According to various embodiments, for example, the pose or position of the robotic system 20 may be known within one second of the acquisition of the official image, within one half second of the acquisition of the fiducial image, within 0.01 seconds of the acquisition of the fiducial image, or any appropriate timing.

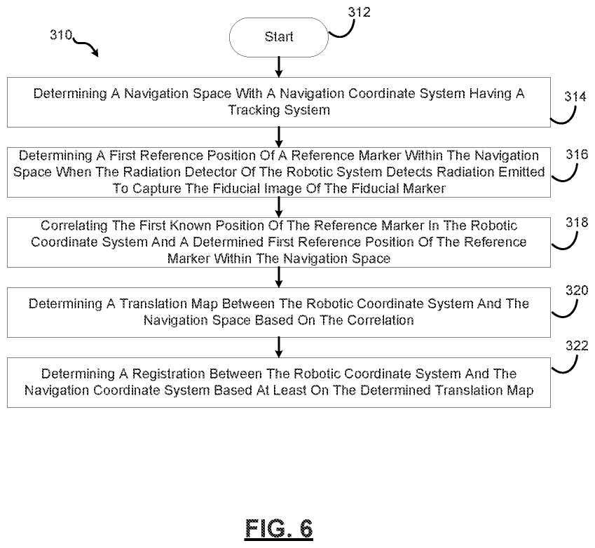

FIG. 6 illustrates a method 310, according to various embodiments, for registering at least two image data, such as a CT to fluoroscopic imaging registration, which may be a continuation of the method 210. The two image data may be a first image data and a second image data. The first image data may precede the second image data in time. The second image data may be acquired with an intraoperative imaging system such as a C-arm imaging system, the O-arm imaging system, etc. The method 310 may be performed by the processor 102, or any other suitable processor. Thus, the following description of the method 310 as being performed by the processor 102 is for exemplary purposes only. The method 310 starts at block 312 and proceeds to block 314. At block 314, the processor 102 determines a navigation space 170 of a navigation coordinate system having a tracking system. The coordinate system of the navigation system may be based on a navigation space of a selected localizer and may also be referred to as patient spaced. Generally, the navigation coordinate system may define a physical space in which the subject 30 may move and/or be placed and in which the robotic system 20 may move and/or be placed. The tracking system may include the localizer and any appropriate tracking device, such as the tracking device 66, 160. The tracking device may have a pose that is determined in physical space due to at least the localizer 88.

At block 316, the processor 102 determines a first reference position of a reference marker within the navigation space when the radiation detector 50 of the robotic system 20 detects radiation emitted to capture the fiducial image of the fiducial marker 140. The reference marker may be any appropriate reference marker, such as a reference marker (e.g., tracker 54) associated with robotic system 20 and/or or a reference marker (e.g., 154) associated with the subject 30. Regardless, the determination of a position of the reference marker may be used to determine a pose of one or more portions during the acquisition of the image data when acquiring the fiducial image. This may allow the tracked pose of the robotic system to be used during the registration process to ensure that the robotic system is at a known pose relative to the subject 30 during a tracked procedure. This may allow for a tracked pose of the robotic system and the subject to be correlated to one another and also correlated to the fiducial image to allow for registration to the pre-acquired image. As discussed above, however, the robotic system 20 may be held relative to the subject 30 with the holding member 21. As the holding member may fix a portion of the robotic system 20 relative to the subject 30, a pose of the robotic system 20 relative to the subject 30 may be determined at any appropriate time. Further, as the reference marker 54 connected to the robotic system 20 is tracked, its tracked pose may be related to the subject 30. Thus, the tracking device 54 may be the only tracking device and the patient tracker 154 may not be required. It is understood, however, that the patient tracker 154 may also be tracked alternatively to the tracking device 54 or in addition thereto. Regardless, the tracked pose of at least a portion of the robotic system 20 may be determined as may a tracked pose of the patient 30 with the tracking device 54 alone and/or in combination with the patient tracker 154.

As discussed above, the fiducial image may be used to correlate the pose of the robotic system 20 to the subject 30 and/or to any pre-acquired image data. As discussed above, registration of the fiducial image (e.g., 2D image) may be made to the pre-acquired image (e.g., 3D image) based upon the acquisition of image date of the subject 30. For example, a selected vertebra may be acquired in both the fiducial image and the pre-required image. Thus, the pre-required image and the fiducial image may be registered based upon points determined of the commonly imaged vertebral portion. The fiducial 140 imaged in the fiducial image may be used to determine a pose of the robotic system, or at least a position of the base 34, relative to the subject 30 based upon the image of the fiducial portion 140. Thus, the robotic system 20 may have its pose or origin coordinated with of the coordinate system of the pre-acquired image and the subject 30 for use during a tracked procedure using the robotic system 20.

At block 318, the processor 102 correlates the first known position of the reference marker in the robotic coordinate system, and a determined first reference position of the reference marker within the navigation space. In other words, the navigation system may track a pose of one or more reference markers at a selected time. For example, the reference marker 154 associated with the subject 30 may be tracked and the reference marker 54 associated with the robotic system 20 may also be tracked. The reference marker of the robotic system 20 may be in the robotic coordinate system. The reference marker on the subject 30 may be in the navigation space coordinate system which may also be defined relative to the subject 30. As the sensor 50 may sense of the time of acquisition of the fiducial image, the same time may be used to track a pose of both of the reference markers 54, 58 associated with robotic system 20 and the subject 30. Thus, the two tr4acked poses of the two reference markers 54, 58 may be used to correlate or determine a translation map of the robotic coordinate system and the navigation coordinate system (e.g., defined by the subject 30) for a correlation or determination of a translation map between the robotic coordinate system and the subject or navigation coordinate system.

At block 320, the method 310 determines a translation map between the robotic coordinate system and the navigation space based on the correlation. The translation map may include a translation or a correlation of points in the coordinate space of the robotic system and navigation system coordinate system. This may be based upon the tracked pose of the reference markers 54, 58. For example, each of the reference markers may include at least one point. The reference markers may, therefore, be used to determine a common point or at least points that may be related to each other in the respective coordinate systems. Further, as noted above, the tracking of the two reference markers 54, 58 may be done substantially simultaneously and/or instantaneously. The simultaneous acquisition of the tracked pose of the referenced markers 54, 58 may relate to the acquisition of the fiducial image to assist in registration or correlation of the coordinate systems of the navigation space and the robotic coordinate system to the pre-acquired image space, at least as discussed above.

At block 322, the processor 102 determines a registration between the robotic coordinate system and the navigation coordinate system based at least on the determined translation map. Generally, the registration may occur based upon the translation map generated by the determination or tracking of common or related points in two coordinate systems. The registration may refer to the status or creation of the translation map. Generally, the translation map may be used to determine a translation between two coordinate systems when a pose in one coordinate system is known or determined, and a determination of a pose in a different coordinate system is required or selected. Therefore, the registration may be made based upon the translation map and may be saved for recall by the processor 102.

Therefore, the registration or translation of the robotic system 20 and its respective coordinate system relative to the subject 30 may be made, as discussed above. The fiducial image may acquire an image of the subject 30 and the fiducial 140 at substantially the same time and include portions of the subject 30 and the fiducial 140 in the same image or image data. The fiducial image that includes an image of a portion of the fiducial 140 and the subject 30 may be used to register or generate a translation map between the fiducial image and any pre-acquired image, such as of the subject 30. The translation map between the fiducial image strand of the pre-acquired image may allow for registration between the two images.

Due to the registration between the two images, the pose of the fiducial 140 may be determined relative to a pre-acquired image. This may allow for a pose of the robotic system 20 to be determined relative to the pre-required image. Thus, any planning that may have occurred in the pre-acquired image may be translated or registered to the current pose of the robotic system 20 relative to the subject 30. The fiducial image that is inclusive of the image of the fiducial 140 and the subject 30 may be, therefore, registered to the pre-acquired image for us during a procedure using the robotic system 20 and relative to the subject 30.

The sensor 50 may allow for a substantially instantaneous determination of the acquisition of the fiducial image. Due to the substantially instantaneous determination of the acquisition of the fiducial image, a movement of the robotic system 20 after the acquisition of the fiducial image may be substantially limited or eliminated. Therefore, the fiducial image may be substantially accurate including the pose of the robotic system at the time of the acquisition of the fiducial image that is used for registration or translation to the pre-acquired image and/or the subject 30.

The foregoing description of the embodiments has been provided for purposes of illustration and description. It is not intended to be exhaustive or to limit the invention. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the invention, and all such modifications are intended to be included within the scope of the invention.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A system operable to register a robotic coordinate system and an image coordinate system, comprising:
a robotic system having the robotic coordinate system defined relative to a first portion of the robotic system, wherein the robotic system is configured to be positioned relative to a subject;
a detector on or proximate to the robotic system;
a tracking system defining a navigation space having a navigation coordinate system;
a fiducial marker at a first known position in the robotic coordinate system relative to the first portion of the robotic system; and
a processor system operable to:
acquire a fiducial image at an initial time with an imaging system of the fiducial marker when the fiducial marker is at the first known position relative to the robotic coordinate system and relative to the first portion of the robotic system;
determine the initial time of the acquisition of the fiducial image with the imaging system with the detector; and
determine or record an initial position of at least one of the first portion of the robotic system or the robotic coordinate system at the initial time.

2. The system of claim 1, further comprising:
the imaging system configured to generate an image the subject and the fiducial marker.

3. The system of claim 2, wherein the imaging system is a x-ray imaging system.

4. The system of claim 1, wherein the detector automatically sends a signal to the processor system upon detection of acquisition of the fiducial image.

5. The system of claim 1, wherein the detector is a radiation detector.

6. The system of claim 1, wherein the detector is a radiation detector on an arm of the robotic system.

7. The system of claim 1, wherein the detector is a radiation detector proximate to an end effector of the robotic system.

8. The system of claim 1, wherein the processor system is further configured to:
determine the navigation space with the navigation coordinate system having the tracking system;
determine a first reference position of a reference marker within the navigation space when the detector of the robotic system detects radiation emitted to capture the fiducial image of the fiducial marker;
correlate the first known position of the reference marker in the robotic coordinate system and a determined first reference position of the reference marker within the navigation space;
determine a translation map between the robotic coordinate system and the navigation space based on the correlation; and
determine a registration between the robotic coordinate system and the navigation coordinate system based at least on the determined translation map.

9. The system of claim 1, wherein the robotic system further includes:
a second portion of the robotic system movable relative to the first portion; and
a snapshot tracking device removably fixed relative to the second portion;
wherein the processor system is further operable to determine a position of the snapshot tracking device in the navigation space; and
wherein the robotic system includes a robotic processor operable to determine a position of the second portion in the robotic coordinate system.

10. The system of claim 1, further comprising a display device configured to display a graphical representation of at least a portion moved with the robotic system based at least on the registration.

11. A method for registering a robotic coordinate system defined by a robotic system and a navigation coordinate system defined by a tracking system localizer, comprising:
determining a robotic coordinate system between a first portion of the robotic system and a subject, wherein the subject is separate from the first portion of the robotic system;
connecting a fiducial marker at a first known position relative to the robotic coordinate system and relative to the first portion of the robotic system;
acquiring a fiducial image at an initial time with an imaging system of the fiducial marker;
detecting acquisition of the fiducial image with the imaging system at the initial time with a detector; and
sending a command to determine or record an initial position of at least one of the first portion of the robotic system or the robotic coordinate system at the initial time.

12. The method of claim 11, wherein the sending the command to determine or record the initial position of at least one of the first portion of the robotic system or the robotic coordinate system at the initial time occurs automatically upon the detecting the acquisition of the fiducial image with the imaging system at the initial time with the detector.

13. The method of claim 11, wherein the detector is a radiation detector.

14. The method of claim 11, wherein the detector is a radiation detector on an arm of the robotic system.

15. The method of claim 11, wherein detecting the acquisition of the fiducial image with the imaging system at the initial time with the detector includes detecting radiation from the imaging system.

16. The method of claim 11, further comprising:
determining a navigation space with the navigation coordinate system having the tracking system;
determining a first reference position of a reference marker within the navigation space when the detector of the robotic system detects radiation emitted to capture the fiducial image of the fiducial marker;
correlating the first known position of the reference marker in the robotic coordinate system and a determined first reference position of the reference marker within the navigation space;
determining a translation map between the robotic coordinate system and the navigation space based on the correlation; and
determining a registration between the robotic coordinate system and the navigation coordinate system based at least on the determined translation map.

17. A method for registering a robotic coordinate system defined by a robotic system and a navigation coordinate system defined by a tracking system localizer, comprising:
determining a robotic coordinate system between a first portion of the robotic system and a subject separate from the first portion of the robotic system;
connecting a fiducial marker at a first known position relative to the robotic coordinate system and relative to the first portion of the robotic system;
acquiring a fiducial image of the fiducial marker at an initial time with an imaging system;
detecting acquisition of the fiducial image with the imaging system at the initial time with a radiation detector of the robotic system configured to detect radiation from the imaging system;
sending a command to determine or record an initial position of at least one of the first portion of the robotic system or the robotic coordinate system at the initial time;
determining a navigation space with the navigation coordinate system having the tracking system;
determining a first reference position of a reference marker within the navigation space when the radiation detector of the robotic system detects radiation emitted to capture the fiducial image of the fiducial marker;
correlating the first known position of the reference marker in the robotic coordinate system and a determined first reference position of the reference marker within the navigation space;
determining a translation map between the robotic coordinate system and the navigation space based on the correlation; and
determining a registration between the robotic coordinate system and the navigation coordinate system based at least on the determined translation map.

18. The method of claim 17, wherein the radiation detector is a radiation detector on an arm of the robotic system.

19. The method of claim 17, wherein the radiation detector is a radiation detector proximate to an end effector of the robotic system.

20. The method of claim 17, wherein sending the command to determine or record the initial position of at least one of the first portion of the robotic system or the robotic coordinate system at the initial time occurs automatically upon the detecting acquisition of the fiducial image with the imaging system at the initial time with the radiation detector.
